(19) United States
(12) Patent Application Publication
(10) Pub. No.: US 2011/0270092 A1
(43) Pub. Date: Nov. 3, 2011

(54) COMBINED APPARATUS FOR DETECTION OF MULTISPECTRAL OPTICAL IMAGE EMITTED FROM LIVING BODY AND FOR LIGHT THERAPY

(75) Inventors: Uk KANG, Gyeonggi-do (KR); Soo Jin Bae, Gyeonggi-do (KR); Guang Hoon Kim, Busan (KR); Seung Yup Lee, Gyeonggi-do (KR); Garry V. Papayan, St. Petersburg (RU)

(73) Assignee: Korea Electrotechnology Research Institute, Gyeongsangnam-do (KR)

(21) Appl. No.: 13/016,642

(22) Filed: Jan. 28, 2011

(30) Foreign Application Priority Data
Jan. 29, 2010 (KR) 10-2010-0008286

(51) Int. Cl. A61B 6/00; A61N 5/06

(52) U.S. Cl. 600/476; 607/88

(57) ABSTRACT

The present invention provides a fluorescence detection and photodynamic therapy apparatus including: a combined light source unit 10 including a plurality of coherent and non-coherent light sources 11, 12 and 13 configured to irradiate light onto a to-be-observed object while performing continuous illumination; an optical imaging unit 20 configured to form an image of the to-be-observed object 70 and project the image to an image processing/controlling system 34; a multispectral imaging unit 30 including a one-chip multispectral sensor and the image processing/controlling system 34; a blocking filter 40 installed between the to-be-observed object 70 and the one-chip multispectral sensor 32, the blocking filter being configured to block some light reflected off from the to-be-observed object 70 while allowing some light and fluorescent light to pass therethrough; a computer system 50 configured to process, analyze, reproduce and store the image acquired from the multispectral imaging unit 30, and transfer the image to a display device 60 and control the overall operation of all the related elements; and the display device 60 configured to display a processing result of the image by the computer system 50.

### CROSS-REFERENCE TO RELATED APPLICATION

This application claims the benefit under 35 U.S.C. §119 (a) of Korean Patent Application No. 10-2010-0008286 filed in the Korean Intellectual Property Office Jan. 29, 2010, the entire contents of which are incorporated herein by reference.

### BACKGROUND OF THE INVENTION

(a) Field of the Invention

The present invention relates to an apparatus for detection of an image emitted from a living body and for light therapy. In particular, it relates to a combined apparatus for detection of a multispectral optical image emitted from a living body and for light therapy useful in a biomedical imaging field, wherein a combined light source illuminates a target animal so that images of a fluorescent light and a reflected light or several fluorescent lights emitted in an in-vivo or in-vitro experiment of the tissues of a target animal are observed and recorded simultaneously in-real time.

(b) Background Art

For the purpose of the research on a fluorescence phenomenon for diagnosis and treatment of various kinds of biomedical diseases, a preclinical research was performed on a living animal, followed by clinical researches and clinical trials.

A fluorophore is mostly generated endogenously or exogenously. Examples of the endogenous fluorophore include collagen, elastin, keratin, NADH, flavin, porphyrin, etc.

The fluorophores contribute to an autofluorescence phenomenon, and the physiological function state of biological organs and systems can be monitored through detection and evaluation of autofluorescence of biological tissues to diagnose diseases such as a tumor.

A fluorescent drug such as a photosensitizer can be externally administered into a living body, and the administered photosensitizer is selectively accumulated at a high concentration in malignant tumor tissues. When an excited light of a specific wavelength is irradiated onto the accumulated region, the position and boundary of an affected tumor can be observed fluorescently by a fluorescent light emitted from the accumulated region.

In addition, singlet oxygen is generated by the photosentization reaction between the photosensitizer and the excited light at the tissue region where the photosensitizer is accumulated, and the generated singlet oxygen destroys tumor cells by a photodynamic therapy without using any surgical means.

On the contrary, an angiography is a medical imaging technique used for observing a state in which a fluorescent photosensitizer injected into a blood vessel is circulated therein. Thus, delay or disorder of blood flow circulation, morphological abnormalities of blood vessels, etc., can be detected. By angiography, it is possible to locate a site of thrombosis, and all the retina disease targets, in particular, an abnormal feature appearing in an acular fundus can be easily detected.

Further, a fluorescence molecular imaging method using the fluorescent photosensitizer enables to observe a topical position of a specific substance with biological importance, measure the amount of the substance, and control delivery of a drug.

In particular, since a near-infrared wavelength penetrates biological tissues much deeper than an ultraviolet ray wavelength and a visible light wavelength do, a fluorescent photosensitizer needs to be highlighted which emits a fluorescent light in the near-infrared wavelength range.

Thus, an apparatus for acquiring a fluorescence image has frequently used monochrome image sensor having sensitivity in the visible light and near-infrared wavelength ranges. In this case, fluorescence excitation is performed by a light source emitting a single wavelength.

U.S. Patent Application No. 2009/0203994 (entitled “Method and apparatus for vasculature visualization with applications in neurosurgery and neurology”, Gurpreet Mangat et al.) and WO2008/070269 (entitled “Methods, Software and systems for imaging”, Brzozowski et al.) disclose a system which is configured to visualize a vasculature and a blood vessel injury position during a surgery.

The above patent documents will be discussed briefly hereinafter.

The conventional systems are primarily characterized by using an angiography method in which indocyanine green as a photosensitizer that is excited to emit a fluorescent light in the near-infrared wavelength range is administered into a blood vessel. By using a laser as a light source for fluorescence excitation and for using a monochrome television camera that provides an image in the form of a black-white frame for the purpose of imaging a blood vessel from which a fluorescent light is emitted.

The above prior art system is unable to simultaneously capture a near-infrared image and a visible light image, and employs a common single light source as a fluorescence excitation light source to implement a system capable of simultaneously recording several fluorescence images whose emitted wavelengths are different from each other. In addition, the conventional system adopts a method of using several monochrome image sensors or dividing a single monochrome image sensor into several image detecting zones to detect several fluorescence images emitting different wavelengths.

U.S. Pat. No. 5,590,660 (Calum MacAulay et al., “Apparatus and methods for imaging diseased tissue using integrated autofluorescence”, 1997) discloses a technology that images diseases in a biological tissue by detecting autofluorescence of the biological tissue. In the above, the imaging apparatus employs a single light source for effecting fluorescence excitation and two monochrome image sensors for detecting fluorescence images produced over two different wavelength bands of fluorescence, in which two filters for passing red and green lights are positioned in front of the two image sensors, respectively.

U.S. Patent Application No. 2008/0051664 (entitled “Autofluorescence detection and imaging of bladder cancer realized through a cystoscope”) discloses an autofluorescence detection apparatus that is used along with an endoscope to conduct fluorescence diagnosis of internal organs of a living body in a near-infrared wavelength range, inter alia, to image bladder cancer through a cystoscope. The above autofluorescence detection apparatus is characterized in that illumination is separately performed on biological tissues using lamps and laser light sources under different light guides, in which case laser light illumination is used for fluorescence excitation and may be performed alternatively from several lasers. For example, a helium-neon laser (oscillated wavelength: about 630 nm) and a Nd:YAG diode-pumped solid-state laser (oscillated wavelength: 532 nm) may be selected.

Moreover, in yet another example of the prior art, a lamp light source can be used to acquire an image from diffused reflection light, a single monochrome image sensor detecting light in a wavelength range of from 650 nm to 1500 nm can be used as a detector, and a band-pass filter can be mounted in front of the detector to select a wavelength. In addition, it was proposed that different portions of a single sensor may be used to simultaneously detect two fluorescence images at different spectral bands. However, the above has the following drawbacks. In the case where white light is irradiated onto an object to be observed (hereinafter, referred to as “to-be-observed object”), color video observation of the to-be-observed object is impossible. Also, it is impossible to conduct multispectral image detection for simultaneously detecting lights in both visible light and near-infrared spectral bands. Further, in the case where light irradiation is performed to transfer light through two different light guides, an endoscopic tool channel needs to be utilized and light irradiation is effected which makes a field of view ununiform, so that the tool channel makes necessary works difficult to be done.

In the meantime, it is essential to obtain a general color image to provide information on morphological features of a biological tissue region observed along with the fluorescence image.

In general, when light is irradiated onto a to-be-observed object using a white-light source, a color image is formed through a reflected light. A variety of methods have been used to simultaneously form a fluorescence image and a general image. As one example of the above methods, U.S. Ser. No. 12/473,745 (Kang, Papayan) use a two-chip TV camera in which a color image sensor is used to detect a general image and a monochrome image sensor is used to detect a fluorescence image in far-red and near-infrared wavelength ranges.

Alec M. De Grand and John V. Frangioni (An Operational Near-Infrared Fluorescence Imaging System Prototype for Large Animal Surgery/Technology in Cancer Research & Treatment. Volume 2, Number 6, December (2003)) proposed a fluorescence imaging system in which indocyanine green (ICG) is intravenously injected to an animal and then a surgery process is observed by an angiography method.

The fluorescence imaging system proposed in the above paper is characterized by using a near-infrared light source emitting light in a wavelength range of from 725 nm to 775 nm and a white-light source emitting light in a wavelength range of less than 700 nm as two light sources for light irradiation, by adopting color and monochrome near-infrared cameras to allow an image of the to-be-observed object to be formed by two independent TV cameras using a zoom lens, by using a dichroic mirror (785 nm center wavelength) for separation of an initial image, and by allowing two video signals formed by the cameras to be inputted to a computer through a frame grabber.

However, the above fluorescence imaging system has a problem that it cannot be used in a light delivery system using an endoscope. Further, it is difficult for images of two independently-operated cameras to be matched with each other spatially or temporarily.

To solve the above problem, U.S. Patent Application No. 2008/0239070 (entitled “Imaging system with a single color image sensor for simultaneous fluorescence and color video endoscopy”, Westwick, Potkins, Fengler, Novadaq Technologies Inc.) discloses a multi-mode light source of an endoscopic imaging system including a single color image sensor for video endoscopes for simultaneous fluorescence and color imaging, as a technology using a single color sensor for simultaneous detection of a fluorescence image and a general image.

The above patent document endoscopic imaging system is characterized by including a endoscopic video system using a single CCD color image sensor chip for detecting a fluorescence image and a general color image and for simultaneously displaying the images at video rates; by using a single-chip color sensor operating in an interlace scanning fashion and a CMYG color coding as the image sensor; by continuously illuminating the a living tissue under investigation with fluorescence excitation light, and periodically illuminating the tissue with the illumination visible light in frequency synchronization with video frame rates of the camera; by disposing an excitation light blocking filter in front of the image sensor to block the excitation light while allowing the blue, green and red components of the illumination light to pass to the color image sensor without interference; by detecting fluorescence images during a time period when only the excitation light is supplied as illumination, and imaging the combination of both tissue fluorescence and reflected illumination light using the color image sensor during a time period when the combination of both the excitation light and the illumination visible light emitted from two light sources are supplied as illumination; by projecting full-frame fluorescence and white-light images onto the image sensor having the interlace scanning fashion; and by subtracting from each full frame of a combined image (fluorescence+color image) a corresponding fluorescence frame image on a pixel-by-pixel basis to produce a real-time fluorescence and white-light images of the living tissue, in which case four full-frame white-light images and two full-frame fluorescence images may be generated every six cycles, and during a cycle where no full frame white-light image is produced, an interpolated image data may be calculated from two adjacent full frame white-light images.

As discussed above, two sensors or a single sensor needing temporal division of an image field are required to simultaneously obtain fluorescence and normal light images or two different fluorescence images in real-time. The application of the two sensors makes the system complicated, and the application of the single sensor reduces the system speed.

The information disclosed in this Background of the Invention section is only for enhancement of understanding of the background of the invention and should not be taken as an acknowledgment or any form of suggestion that this information forms the prior art that is already known to a person skilled in the art.

### SUMMARY OF THE INVENTION

The present invention has been made in an effort to solve the above-mentioned problems occurring in the prior art, and it is an object of the present invention to provide a fluorescence detection and photodynamic therapy apparatus having a simple structure in which fluorescence and normal white-light images or two or more fluorescence images can be provided in real-time by a single sensor to supply a multispectral image without any complex image processing works.

In order to accomplish the above object, the present invention provides a fluorescence detection and photodynamic therapy apparatus, including: a combined light source unit 10 including a plurality of coherent and non-coherent light sources 11, 12 and 13 configured to irradiate light onto a to-be-observed object, while performing continuous illumination; an optical imaging unit 20 configured to form an image of the to-be-observed object 70 and project the image to an image processing/controlling system 34; a multispectral imaging unit 30 including a one-chip multispectral sensor and the image processing/controlling system 34; a blocking filter 40 installed between the to-be-observed object 70 and the one-chip multispectral sensor 32, the blocking filter being configured to block some light reflected off from the to-be-observed object 70 while allowing some light and fluorescent light to pass therethrough; a computer system 50 configured to process, analyze, reproduce and store the image acquired from the multispectral imaging unit 30, and transfer the image to a display device 60 and control the overall operation of all the related elements; and the display device 60 configured to display a processing result of the computer system 50.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 is a schematic diagram illustrating a combined apparatus for detection of a multispectral optical image emitted from a living body and for light therapy according to a preferred embodiment of the present invention;

FIG. 2 is a graph illustrating spectral sensitivity in both visible light and near-infrared wavelength ranges of an RGB CCD image sensor applied to the present invention;

FIG. 3 is a schematic view illustrating a Bayer-type color coding RGB CCD image sensor and reaction of the image sensor to white light and near-infrared light;

FIG. 4 is a schematic diagram illustrating an array construction of a combined light source unit including a common light guide of a combined apparatus for detection of a multispectral optical image emitted from a living body and for light therapy according to the present invention;

FIG. 5 is a schematic diagram illustrating the construction for optical observation of a small animal of a combined apparatus for detection of a multispectral optical image emitted from a living body and for light therapy according to the present invention;

FIG. 6 is a photograph showing a prototype of the combined apparatus for detection of a multispectral optical image emitted from a living body and for light therapy according to the present invention;

FIG. 7 is a schematic diagram illustrating the case where a research is conducted in a clinical condition using a combined apparatus for detection of a multispectral optical image emitted from a living body and for light therapy according to the present invention;

FIG. 8 shows multispectral images of a mouse transplanted with TC-1 tumor cells in autofluorescence as the results of a test example of the present invention;

FIG. 9 is graphs illustrating an excitation light condition and a fluorescence detection condition for acquisition of a multispectral image of the present invention;

FIG. 10 illustrates a fluorescence photograph showing an experimental result of a fluorescence angiography using excitation light of 805 nm wavelength together with fluorophore indocyanine green, and a fluorescence photograph showing a color image of the same to-be-observed object acquired through the simultaneous operation of a broad-band light source emitting light having a wavelength range of from 400 nm to 700 nm and a laser excitation light source emitting light of 805 nm wavelength together with fluorophore indocyanine green as the results of a test example of the present invention; and

FIG. 11 is a graph illustrating the evaluation of an effective photo-bleaching effect of a chlorine-based photosensitizer with the aid of a multispectral imaging unit when a light source emitting light with a center wavelength of 405 nm and a 662 nm laser as a light source of the present invention irradiate light onto a biological tissue, respectively.

Reference numerals set forth in the Drawings includes reference to the following elements as further discussed below:
10: combined light source unit
11: first light source
12: second light source
13: third light source
14: common light guide
15: first mirror
16: second mirror
17: focal lens
18: projective lens
19: filter wheel
20: optical imaging unit
22: movable polarizer
24: band-pass filter
30: multispectral imaging unit
32: one-chip multispectral sensor
34: image processing/controlling system
40: blocking filter
42: filter wheel
50: computer system
60: display device
70: to-be-observed object
80: imaging head
82: support
84: vertical support
86: horizontal support
88: moving plate

### DETAILED DESCRIPTION

Reference will now be made in detail to the preferred embodiment of the present invention, examples of which are illustrated in the drawings attached hereinafter, wherein like reference numerals refer to like elements throughout. The embodiments are described below so as to explain the present invention by referring to the figures.

Now, a preferred embodiment of according to the present invention will be described hereinafter in detail with reference to the accompanying drawings such that those skilled in that art to which the present invention pertains can easily carry out the embodiment.

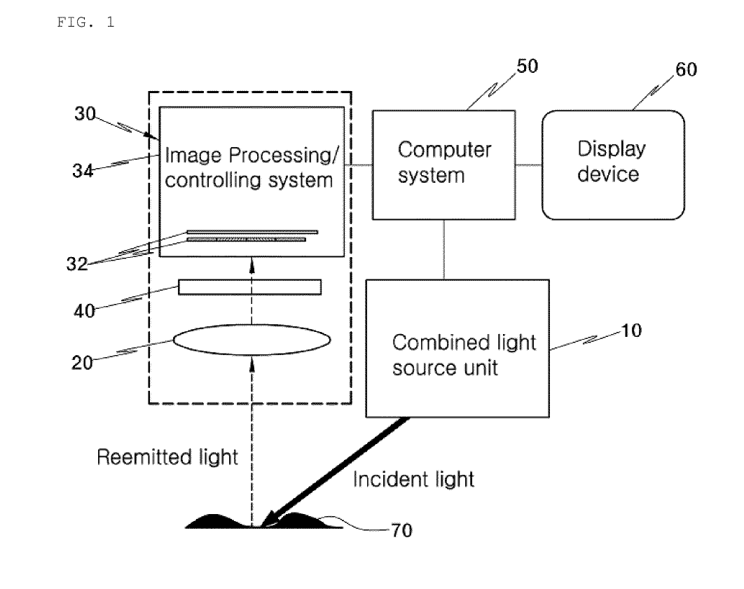

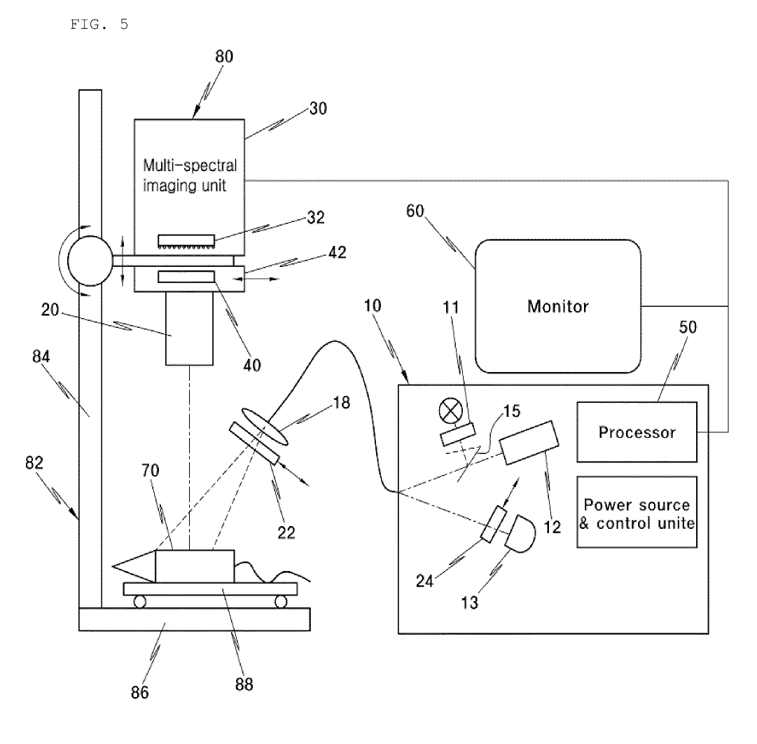

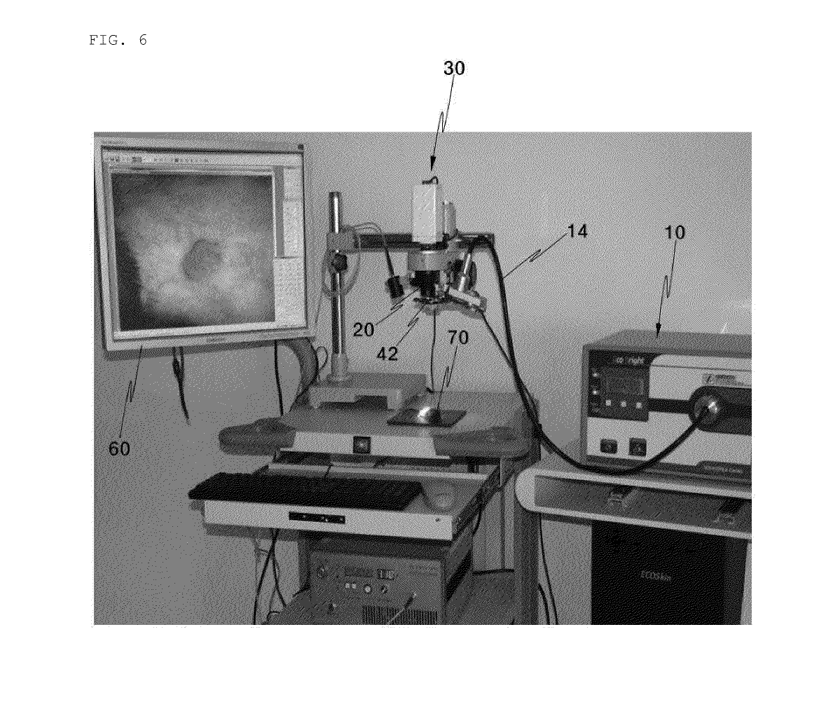

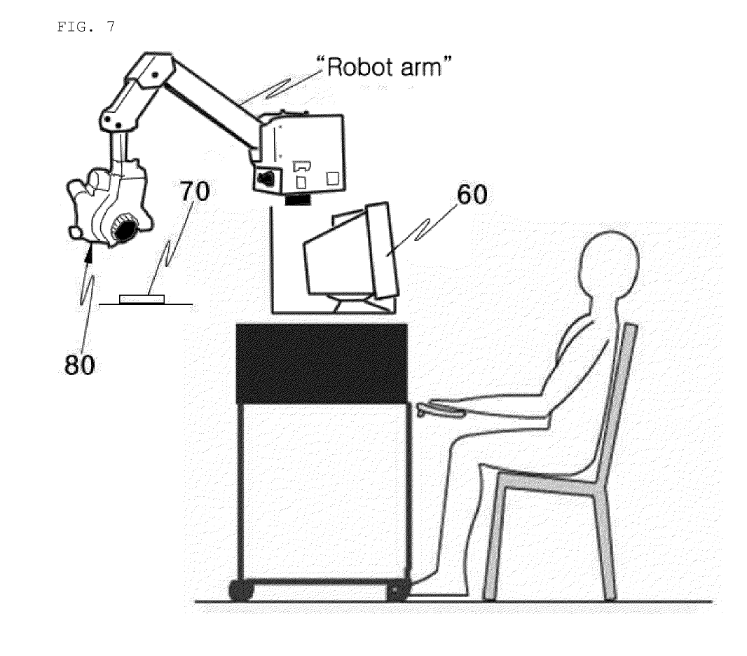

FIG. 1 is a schematic diagram illustrating a combined apparatus for detection of a multispectral optical image emitted from a living body and for light therapy according to a preferred embodiment of the present invention, FIG. 4 is a schematic diagram illustrating an array construction of a combined light source unit including a common light guide of a combined apparatus for detection of a multispectral optical image emitted from a living body and for light therapy according to the present invention, FIG. 5 is a schematic diagram illustrating the construction for optical observation of a small animal of a combined apparatus for detection of a multispectral optical image emitted from a living body and for light therapy according to the present invention, and FIG. 6 is a photograph showing a prototype of the combined apparatus for detection of a multispectral optical image emitted from a living body and for light therapy according to the present invention.

The present invention is aimed to provide a fluorescence detection and photodynamic therapy apparatus for exhibiting morphological and biological characteristics of the biological tissue from fluorescent light and reflected light in order to research a normal or diseased tissue in an in-vivo or in-vitro experimental condition for a biological tissue of a to-be-observed object, and performing both diagnosis and photodynamic therapy.

To this end, the fluorescence detection and photodynamic therapy apparatus of the present invention is characterized in that it includes: a combined light source unit 10 including a plurality of coherent and non-coherent light sources 11, 12 and 13 configured to irradiate light onto a to-be-observed object while performing continuous illumination; an optical imaging unit 20 configured to form an image of the to-be-observed object 70 and project the image to an image processing/controlling system 34; a blocking filter 40 configured to block some light reflected off from the to-be-observed object 70 while allowing some light and fluorescent light to pass therethrough; a multispectral imaging unit 30 including a one-chip multispectral sensor and the image processing/controlling system 34; a computer system 50 configured to receive a signal from the multispectral imaging unit 30 and perform processing operations for the processing, analysis, and reproduction of the image acquired from the multispectral imaging unit 30 in response to the signal; and a display device 60 configured to display a processing result of the computer system 50, whereby a multispectral image is simultaneously formed from fluorescent light and reflected light, or the multispectral image is formed as a color image from two or more fluorescent light such that different wavelengths are used for fluorescence excitation.

Light irradiation is simultaneously performed on a to-be-observed object 70 as a specific site of certain biological organs by a plurality of light sources 11, 12 and 13 included in the combined light source unit 10. The term “light irradiation”, as used herein, refers to electromagnetic radiation. A wavelength range of light for the electromagnetic radiation is classified into a visible light wavelength range (Visible light, VIS, 400-700 nm), a near-ultraviolet wavelength range (UVA, 320-400 nm), and a near-infrared wavelength range (NIR, IR-A: 700-1400 nm).

In addition, the light sources 11, 12 and 13 of the combined light source unit 10 are several coherent and non-coherent light sources for performing continuous illumination. These coherent and non-coherent light sources may be as follows:

1) A white-light source as the first light source 11, which includes a lamp (white LED, halogen lamp, xenon lamp, etc.) emitting a continuous spectrum of light in a visible light wavelength range, and is mounted with a band-pass filter necessarily serving to control the wavelength range of emitted light.

2) A laser light source (laser diode, laser diode array, and fiber pigtailed laser diode) as the second light source 12 emitting monochrome light in a wavelength range of from 400 nm to 900 nm, and

3) A band-pass light source as the third light source 13 including a lamp emitting light in a short wavelength range of from 320 nm to 600 nm, and a band-pass filter having a half-intensity width of 60 nm or less.

In this case, the lamp used in the third light source 13 may use a mercury lamp, an LED, a fiber-pigtailed LED, a xenon lamp, etc.

The first light source 11 of the light sources of the combined light source unit 10 irradiates light onto a to-be-observed object to acquire a normal image from reflected light and polarized light. The second light source 12 and the third light source 13 irradiates light onto the to-be-observed object to effect fluorescence excitation and perform photodynamic therapy simultaneously when a fluorophore exists in a biological tissue.

These light sources may irradiate light onto the to-be-observed object independently, but at least two light sources are required to simultaneously irradiate light onto the to-be-observed object to acquire a combined image.

For example, the first light source 11 and the second light source 12 are simultaneously operated in a reflectance/fluorescence 1 condition, the first light source 11 and the third light source 13 are simultaneously operated in a reflectance/fluorescence 2 condition, and the second light source 12 and the third light source 13 simultaneously operated in a fluorescence 1/fluorescence 2 condition.

Preferably, the first light source 11 is a white-light source emitting light in a wavelength of 400 nm to 700 nm, which may use any one selected from the group consisting of a halogen lamp, a white lamp, an RGB LED, a xenon lamp, and a metal haloid lamp.

In addition, the second light source 12 is a monochrome light source consisting of two laser light sources. The laser light source may use anyone selected from the group consisting of a single laser diode, a plurality of laser diode arrays, and a fiber-pigtailed laser diode, each of which emits monochrome light in a wavelength of from 400 nm to 900 nm.

Further, the third light source 13 is a band-pass light source including a lamp emitting light in a short wavelength range. The band-pass light source may use any one selected from the group consisting of a mercury lamp, an LED, a fiber-pigtailed LED, and a xenon lamp, each of which includes a band-pass filter having a half-intensity width of 60 nm or less in a wavelength range of from 320 nm to 600 nm.

In this case, the light irradiation by the light sources 11, 12 and 13 is performed through a common light guide 14 that is commonly used such as an independent light channel or a liquid light guide.

The common light guide 14 is a liquid light guide, and is a common irradiation path of light emitted from the first light source 11, the second light source 12, and the third light source 13.

Selectively, the second light source 12 and the third light source 13 can irradiate light onto the to-be-observed object through the common light guide 14, and the first light source 11 can irradiate light onto the to-be-observed object 70 directly, but not through the common light guide 14. Alternatively, the second light source 12 and the third light source 13 may irradiate light onto the to-be-observed object 70 through different light guides (for example, a laser light guide using a monofiber light guide). Although not shown, a collimating lens may be additionally installed behind the monofiber light guide to allow light to be irradiated onto a narrower site of the to-be-observed object side.

In this case, when the light sources 11, 12 and 13 irradiate incident light onto the to-be-observed object 70, reflected light is reflected from the to-be-observed object 70 and fluorescence is emitted from the to-be-observed object 70 into an imaging head 80 for formation of a multispectral image.

Herein, the imaging head 80 is a single integral structure in which the optical imaging unit 20 including an objective lens, the blocking filter 40, and the multispectral imaging unit 30 including the one-chip multispectral sensor 32 and the image processing/controlling system 34 are assembled.

An optical imaging system as a constituent element of the imaging head 80, i.e., the optical imaging unit 20 serves to form the images of fluorescence and reflected white-light emitted from the to-be-observed object 70 on the one-chip multispectral sensor 32 of the multispectral imaging unit 30. The optical imaging unit 20 may use an objective lens, an endoscope, a stereo microscope, etc.

Preferably, the optical imaging unit 20 uses an objective lens. The optical imaging unit 20 may use an objective lens having a fixed focal point, an objective lens having a zoom function, an objective lens having an automatic focusing function performed by a motor, etc. In addition, the optical imaging unit 20 preferably uses an objective lens having an aperture stop for controlling the quantity of light and the depth of field.

Moreover, the blocking filter 40 as a constituent element of the imaging head 80 may installed between the optical imaging unit 20 and the to-be-observed object 70, at the inside of the optical imaging unit 20, or between the optical imaging unit 20 and the one-chip multispectral sensor 32 so as to serve to block reflected light which is irradiated onto the to-be-observed object 70 by the second light source 12 and the third light source 13 and then is reflected from the to-be-observed object 70, and transmit reflected light which is irradiated onto the to-be-observed object 70 by the first light source 11 and then is reflected from the to-be-observed object 70 and fluourescence light emitted from the to-be-observed object 70.

In this case, the blocking filter 40 may use a single-band-pass filter, a multi-band-pass filter, a notch filter, and an edge long pass filter. Preferably, the blocking filter 40 is arranged in plural numbers along a circumferential direction within a filter wheel 42 rotatably driven by a given driving source for the rapid exchange of a filter.

The multispectral imaging unit 30 as a constituent element of the imaging head 80 includes a one-chip multispectral sensor 32 and an image processing/controlling system 34.

In particular, preferably, sensor pixels of the one-chip multispectral sensor 32, i.e., sensor pixels having light sensitivity has selective sensitivity in the visible light wavelength range, and simultaneously also have sensitivity for light of a wavelength range out of the visible light wavelength range. An example of the one-chip multispectral sensor 32 having a spectrum with color channels while having such spectral sensitivity may include a One-chip RGB CCD image sensor, a CMOS image sensor, and an EMCCD image sensor.

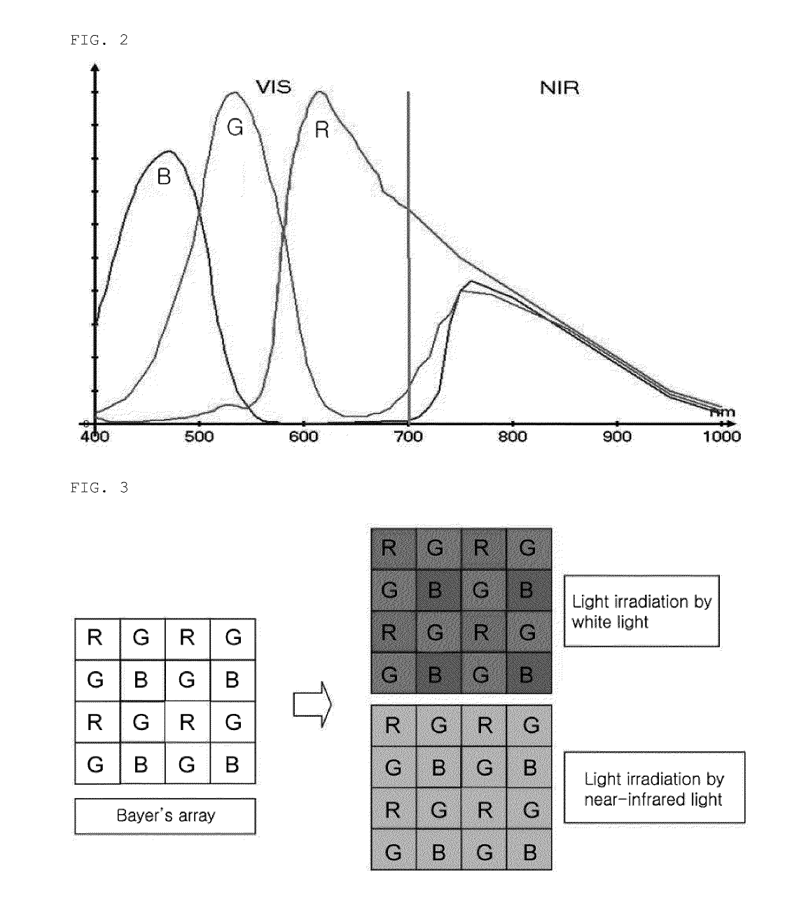

As shown in FIG. 2, the One-chip RGB CCD image sensor 32 is designed in consideration of its light sensitivity characteristics and spectral characteristics of respective filters. The respective pixels of the One-chip RGB CCD image sensor 32 have sensitivity in the R-canal, G-canal, and B-canal spectral ranges by filters arranged in the mosaic shape on a silicon image sensor. Since each of the red, green and blue spectral filters has an additional pass band in a visible light (VIS) wavelength range as well as a near-infrared (NIR) wavelength range, all the pixels have a high light sensitivity in a visible light wavelength range and simultaneously have light sensitivity in the near-infrared wavelength range.

Thus, a near-infrared channel as a fourth spectral channel is formed, so that the spectral sensitivity in the near-infrared wavelength range is mainly determined by sensitivity to light of the silicon image sensor itself and slightly depends on the selectivity characteristics of the red, green and blue spectral filters.

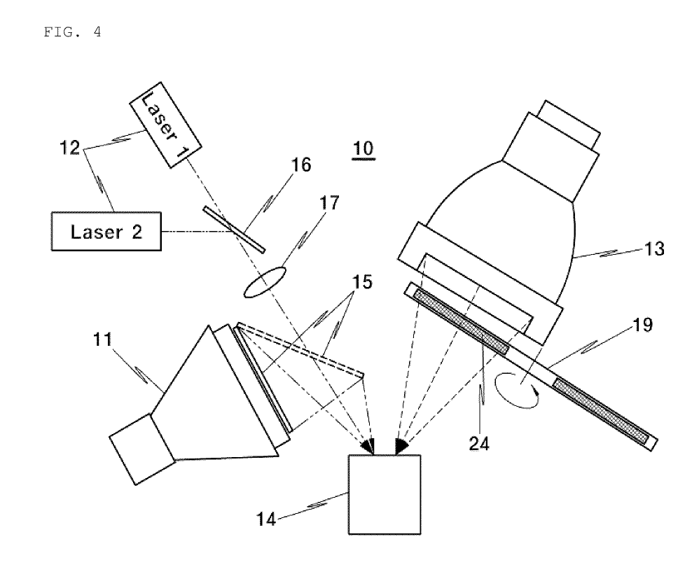

FIG. 3 is a schematic view illustrating a Bayer-type color coding RGB CCD image sensor and reaction of the image sensor to white light and near-infrared light. In FIG. 3, there is illustrated a Bayer-type color coding mask array and reaction of the image sensor to lights with a visible light wavelength range of from 400 nm to 700 nm (white light) and a near-infrared wavelength range of from 750 nm to 1000 nm along with the mask array.

As seen from the left side of FIG. 3, light with a wavelength range of 750 nm or more is detected as achromatic by the color coding RGB CCD image sensor. As seen from the bottom of right side of FIG. 3, extension of light sensitivity to a wavelength range out of a boundary of the visible light wavelength range (400-700 nm), i.e., the near-infrared wavelength range (750-1000 nm) adds an optical signal to all the pixels of the image sensor, which generally causes distortion of color delivery and produces an image with decreased saturation of color.

Therefore, in a general system manufactured to detect an image of visible light, hot mirror type filters for eliminating near-infrared light can be installed in front of the image sensor to reduce damage of an RGB image.

However, in the present invention, a separate hot mirror is not installed in front of the image sensor to block the near-infrared light. The reason for this is that the near-infrared light is used as an important optical signal, but not a noise in a general case, in a fluorescence detection experiment.

In the present invention, a blocking filter (i.e., notch filter) is installed instead of the hot mirror to block light in the narrow wavelength spectral band of excitation light. Of course, the blocking filter can pass therethrough light in the remaining visible light and near-infrared wavelength ranges other than light in the wavelength range of the excitation light, and thus fluorescence image can be detected and recorded in the visible light and near-infrared wavelength ranges.

In the meantime, a surrounding environment is important in confirmation of signals received from the near-infrared channel.

That is, in the case where detected signal light is distributed only in the near-infrared spectral band, since RGB pixels detect only near-infrared light and the image sensor is operated like a monochrome image sensor, the confirmation of the signal is simple. However, there is caused a problem when light in the visible light and near-infrared wavelength ranges is simultaneously irradiated onto the image sensor, for example, when VIS reflectance and NIR fluorescence are detected simultaneously.

In order to address and solve this problem, a series of signal characteristics included in a combined image of the present invention can be taken into consideration or a light irradiation condition can be changed to confirm the near-infrared image even in an image in which visible light and near-infrared light are combined as follows.

1) Increase in Brightness and Change in Color at a Specific Region of a Biological Tissue According to Addition of a Near-Infrared Light Signal

Generally, since near-infrared fluorescence is shown topically only at a specific region, for example, near-infrared fluorescence can be restrictedly observed only at a region where a specific substance is distributed in a fluorescence molecular imaging method, and thus the flow of lymph can be observed in a fluorescence microlymphography.

A relevant observed region can be confirmed owing to high fluorescence brightness and low saturation (white color) of the specific substance as compared to the surrounding biological tissues encircling such a topical region.

2) Distribution of Near-Infrared Fluorescence Existing Only in a Specific Structure of a Biological Tissue

Since this case occurs in a fluorescence angiography method in which near-infrared fluorescence dye ICG is concentrated in a blood vessel, the distribution position of fluorescence dye is readily confirmed through the characteristic image of a vasculature. In addition, the dynamic motion of the fluorescence dye in the vasculature can be observed, and a change in fluorescence image can be timely distinguished to compare changes in anterior and posterior fluorescence images.

Meanwhile, reflected light is observed to be darker by light absorption of hemoglobin of blood vessel tissues, and resultantly a visible light signal at a position where blood vessels are arranged is relatively weak as compared to the surrounding biological tissues.

3) Change in Spectral Component of Reflected Light

If color intensity is insufficient to confirm a to-be-observed object emitting fluorescence in a background of reflected color light, the spectral component of irradiation light can be changed to increase the light-dark intensity. As an example, an image having better brightness can be acquired in light irradiation in which a red spectral component is eliminated to observe chlorine e-6 fluorescence.

4) Change in Light Source Brightness

In the case where it is uncertain whether a given characteristics of an image is caused by visible light or infrared light, one of the light sources of the combined light source can be temporarily turned off to investigate the correlation between the characteristics of an image and the light source.

FIG. 4 is a schematic diagram illustrating an embodiment of a combined light source unit including a common light guide of a fluorescence detection and photodynamic therapy apparatus according to the present invention;

In a concrete embodiment of the combined light source unit 10 according to the present invention, a halogen lamp is used as a white-light source as the first light source 11, and two lasers are used as a monochrome light source as the second light source 12

In addition, a mercury lamp is used to serve as an optical band light source which is the third light source 13. A filter wheel 19 including band-pass filters 24 is positioned in front of the mercury lamp, and a liquid light guide is used as a common light guide 14

In this case, light irradiation toward the common light guide 14 from the halogen lamp of the white-light source as the first light source 11 is performed with the aid of a first mirror 15. The first mirror 15 may use a dichroic mirror or a movable opaque mirror.

In particular, the first mirror 15 is disposed in front of the first light source 11 to allow light emitted from the first light source 11 to be reflected therefrom toward the liquid light guide 14, and is arranged in a structure in which the first mirror can be moved (i.e., angularly rotated) toward the first light source 11 or the second light source 12 by a certain driving means (for example, motor, etc.) to allow light from the first light source 11 and light from the second light source 12 to be alternately irradiated onto the light guide 14.

The turning on and turning off of the light sources is performed depending on the position of the movable first mirror 15 as listed in Table 1 below.

TABLE 1

| Mode | Laser | White lamp | Movable first mirror |
|---|---|---|---|
| White | OFF | ON | B |
| Laser | ON | OFF | A |

In the meantime, a second mirror 16 as the dichroic mirror is fixedly disposed in front of the second light source 12 of the combined light source unit 10 to allow lights emitted from two lasers to be simultaneously irradiated onto the common liquid light guide 14. In addition, a focal lens 17 is further disposed in front of the second mirror 16 to allow lights emitted from the lasers as the second light source 12 to be correctly irradiated onto the common light guide 14.

Besides, a band-pass filter 24 of the band-pass light source as the third light source 13 is either a single band-pass filter or a multi-band-pass filter. The band-pass filter 24 is arranged in plural numbers along a circumferential direction within a disc-like filter wheel 42 rotatably driven by a given driving source to provide rapidness and facilitation of exchange of a filter.

FIG. 5 is a schematic diagram illustrating the construction of the combined apparatus of the present invention included to perform a biomedical research in an in-vivo or in-vitro experimental condition of experimental animal tissues, and FIG. 6 is a photograph showing a prototype of the combined apparatus of the present invention.

As described above, ascendably and descendably installed at a certain support is the imaging head 80 which includes the optical imaging unit 20 including the objective lens and the blocking filter 40, the multispectral imaging unit 30 including the one-chip multispectral sensor 32 and the image processing/controlling system 34.

In this case, the support 82 includes a vertical support 84 assembled allow the imaging head 80 to ascend and descend, and a horizontal support 86 integrally joined at a side thereof to a lower end of the vertical support 84 so that the to-be-observed object 70 is placed on the horizontal support 86.

More specifically, a body portion of the imaging head 80 is ascendably and descendably assembled to the vertical support 84 of a predetermined height extending in a vertical direction, so that the imaging head 80 can be moved in a horizontal direction relative to an optical axis of the imaging head 80 so as to be focused on the to-be-observed object 70 placed on the horizontal support 86.

In this case, the support 82 may include a flat moving plate 88 having a movable means attached to a bottom thereof, so that the to-be-observed object 70 is fixed to the top of the moving plate 88, and then the moving plate 88 is seated on the horizontal support 86. Thus, the movement of the moving plate 88 can be adjusted to easily move the to-be-observed object 70 positioned on the moving plate 88 to a position perpendicular to the optical axis of the imaging head 80.

In addition, a projective lens 18 is installed in front of the liquid light guide 14 to allow light irradiation to be performed by uniformly magnifying light. In addition, when it is desired to perform observation by polarized light, a movable polarizer 22 for operation under a crossed polarized light condition is installed between the light guide and the to-be-observed object. A crossed analyzer is installed in front of the imaging head along with the movable polarizer, so that reflection of polarized light cuts off components of the reflected light of the mirror by the crossed analyzer and allows an image to be acquired from the diffused reflection light.

Further, a computer system 50 is built in a casing of the combined light source unit of the present invention, and a processor of the computer system 50 controls the overall operation of all the elements of the combined apparatus of the present invention, and serves to perform the processing to process, analyze, and reproduce an image.

Of course, the computer system 50 includes an RGB monitor as the display device 60, parts (keyboard, and mouse), and a device for two-way interactivity.

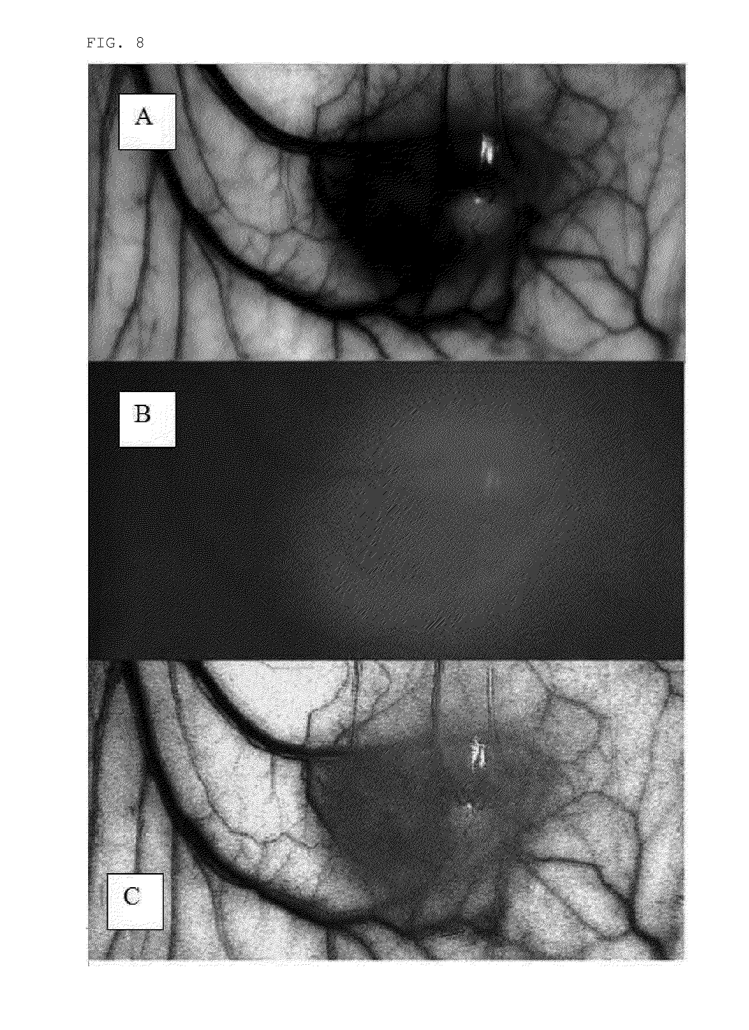

For reference, in the case where a research is conducted under a clinical condition (common operating room in surgery, obstetrics & gynecology, dentistry, etc.), the imaging head of the present invention can be used by being fixed to a movable support such as a robot arm (see FIG. 7).

Herein, the operation of the combined apparatus for detection of a multispectral optical image emitted from a living body and for light therapy will be discussed hereinafter by way of test examples.

FIG. 8 shows multispectral images of a mouse transplanted with TC-1 tumor cells in autofluorescence (4 days after transplantation of tumor cells)

In case of a photograph A of FIG. 8, ultraviolet ray and blue excitation light are generally used for diagnosis of tumor by autofluorescence using a broad-band light source (370-410 nm in wavelength). In this case, an image of a region where a tumor grows is observed to be dark. This for reason is that the amount of fluorescence of a tumor region is reduced by growth of a new blood vessel supplying oxygen and nutrients to the tumor region and light absorption of hemoglobin in blood vessel tissues as a basic diagnosis sign. But this sign is not applied to only the case of the tumor.

In case of photograph B of FIG. 8, a diagnosis method is adopted to locate a tumor using a laser light source (635 nm in wavelength). In a fluorescence diagnosis method using 5-aminolevulinic acid (5-ALA) as a precursor of protoporphyrin (PpIX), when 5-ALA as a total synthesis substance of porphyrin is applied to a biological organ, 5-ALA at the tumor region is increased in concentration while being converted into protoporphyrin (PpIX) as a fluorophore in tumor cells. As shown in the photograph B of FIG. 8, red spectral fluorescence can be detected to readily locate the tumor. However, a drawback of this fluorescence diagnosis method resides in that the photosensitizing agent is necessarily applied from the outside of the biological organ.

In addition, in order to detect a porphyrin flourescence signal through the optical method, it is required that protoporphyrin (PpIX) be excited by laser light irradiation near 635 nm. As shown in the photograph B of FIG. 8, single laser light irradiation does not provide information on the morphological structure of biological tissues.

Alternatively, light irradiation [(370-410 nm)+635 nm] by two light sources is used to obtain the advantages of the above-mentioned two methods in the present invention. In a test example 1 of the present invention, to acquire images of autofluorescence 1/autofluorescence 2, the second light source 12 and the third light source 13 of the combined light source unit 10 are operated to irradiate light emitted from a 635 nm laser and a narrow-band light source.

In the present invention, two excitation light sources irradiate light onto the biological tissue as the to-be-observed object simultaneously, i.e., irradiate excitation lights having wavelengths of 390±40 nm and 635 nm onto the biological tissue simultaneously, so that a multispectral image of the tumor is seen as shown in a photo graph C of FIG. 8.

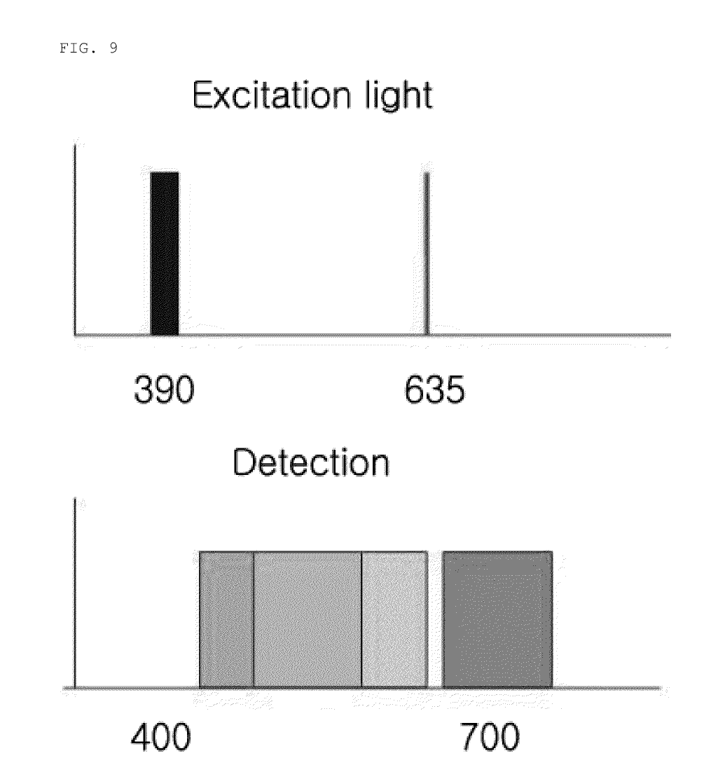

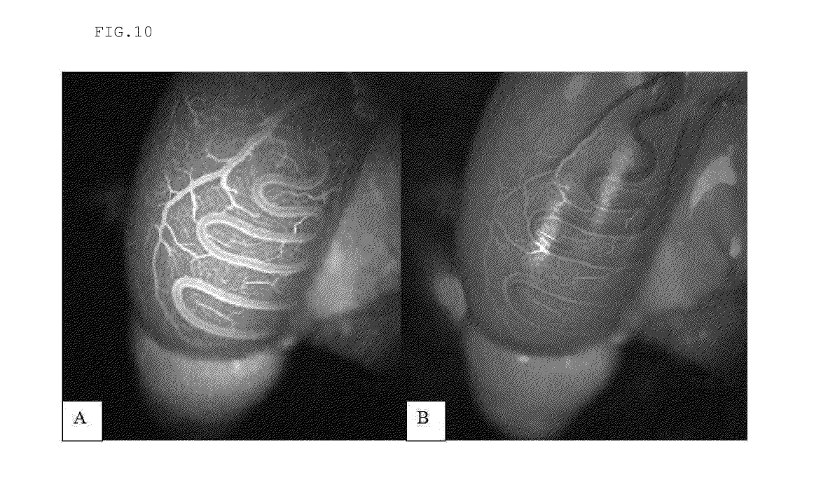

FIG. 9 is graphs illustrating an excitation light condition and a fluorescence detection condition for acquisition of a multispectral image of the present invention.

Fluorescence signals of the blue (B) and green (G) channels are mainly determined by NADH and flavin, and a fluorescence signal of a red (R) channel is determined by PpIX. In this case, since a hot mirror reflecting near-infrared light is not positioned in front of the sensor, a fluorescence signal with a spectral band of from 650 nm to 750 nm emitted from PpIX can be detected.

In this case, the blue (B) and green (G) channels provide information on an oxidation-reduction reaction of the biological tissue and information on the morphological structure of a vasculature. The red (R) channel provides information on the position and proliferation intensity of a tumor improve sensitivity and specificity simultaneously in the diagnosis of tumor diseases.

In a test example 2 according to present invention, a 808 nm laser and a broad-band light source are used to acquire images of near-infrared (NIR) fluorescence/white reflected light.

A fluorophore (for example, indocyanine green, ICG) emitting fluorescence in a near-infrared spectral band has been widely in the biomedical research. Fluorophores are used to trace the flow of blood and lymph in order to locate a topical site where a specific substance which it is desired to observe is distributed in the molecular imaging method using fluorescence angiography and lymphangiography.

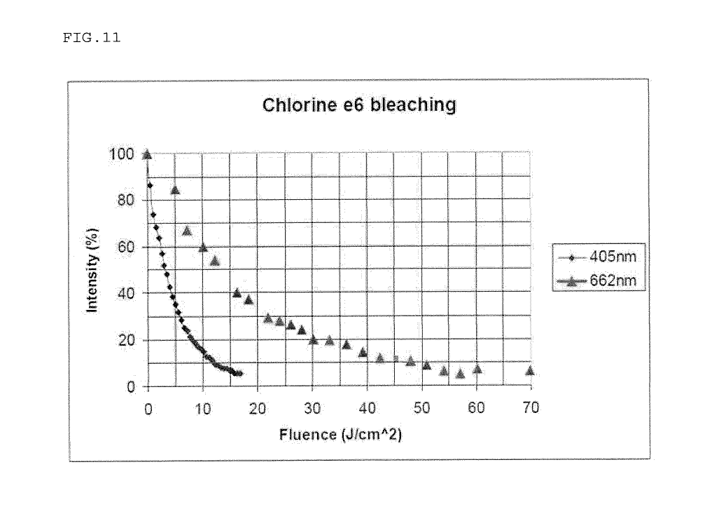

In the imaging system proposed in the above-mentioned U.S. Patent Application No. 2009/0203994 and PCT International Patent Publication No. WO2008/070269, a 805 nm laser is used as the excitation light source, and a monochrome camera is used as the image sensor. Such an imaging system shows a near-infrared monochrome image only, but not the near-infrared monochrome image and a normal color image simultaneously (see photograph A of FIG. 10).

For reference, a photograph A of FIG. 10 is an experimental result of a fluorescence angiography using excitation light of 805 nm wavelength together with fluorophore indocyanine green, which shows a black-white monochrome image of an animal's testis by the near-infrared fluorescence.

Alternatively, according to the test example 2 of the present invention, a color image of the same to-be-observed object can be acquired through the simultaneous operation of a broad-band light source emitting light having a wavelength range of from 400 nm to 700 nm and a laser excitation light source emitting light of 805 nm wavelength together with fluorophore indocyanine green as shown in a photograph B of FIG. 10.

That is, in the combined apparatus according to the present invention, a 805 nm notch filter as the blocking filter, abroad-band light source emitting light having a wavelength range of from 400 nm to 700 nm and a laser excitation light source emitting light of 805 nm wavelength can be simultaneously operated to simultaneously acquire a color image and a near-infrared image.

As a result, as shown in the photograph B of FIG. 10, a bright portion formed by fluorescence emitted by indocyanine green is observed along a boundary of a blood vessel in a background of a normal color image of the biological tissue.

In this case, a surrounding bright spot in the photograph B of FIG. 10 is caused by reflected light and can be removed under the polarized light condition. Since indocyanine green is distributed in only a blood vessel, it is not difficult to identify the near-infrared image in the blood vessel.

In a test example 3 of the present invention, a photodynamic therapy method by a fluorescence bleaching and a white reflected light image is used. The method is characterized in that a 650-660 nm laser and a red-free light source are used.

The photodynamic therapy is a method which is effective in treatment of various diseases. A broad-band light source can be used together with a laser as a therapy light source for the photodynamic therapy.

In this case, one of factors that are important in performing the photodynamic therapy is the amount of light irradiated. Alight irradiation amount for therapy can be adjusted by checking a degree of occurrence of a whitening phenomenon during light irradiation.

The adjustment of the light irradiation amount for the photodynamic therapy can be performed by the use of the multispectral imaging unit, and a fluorescence bleaching phenomenon curve using a chlorine e6-based photosensitizer fluorophore is shown in a graph of FIG. 11.

FIG. 11 is a graph illustrating a result of the evaluation of an effective photo-bleaching effect of a chlorine-based photosensitizer using the multispectral imaging unit when a light source emitting light with a center wavelength of 405 nm and a 662 nm laser irradiates light onto a biological tissue, respectively.

The light irradiation for the biological tissue was stopped when the bleaching phenomenon reaches a predetermined level. For example, when fluorescence intensity is decreased 10 times, the light irradiation is suspended. In addition, the structure and morphological characteristics of the biological tissue to which light is irradiated needs to be observed to confirm whether or not therapy light is correctly irradiated onto a topical region along with fluorescence observation.

In the meantime, a red spectral component can be eliminated and the light irradiation can be adjusted under a given control condition to increase the intensity of a color image when light irradiation by a broad-band light source is performed. This method enables light irradiation to be suspended when reaching a predetermined level of photo-bleaching effect while easily confirming the regions where light irradiation is performed, which resultantly becomes a factor of effectively performing a photodynamic therapy process.

The present invention provides the following effects through the above problem solving means.

As described above, according to of the present invention, fluorescence and normal white-light images or two or more fluorescence images for a specific region of a biological tissue as a to-be-observed object are provided as color images in real-time using a plurality of different light sources and a one-chip multispectral sensor, thereby more effectively performing a photodynamic observation and therapy.

That is, the present invention can provide a research means capable of elucidating morphological and biological characteristics of the biological tissue from fluorescent light and reflected light in order to research a normal or diseased tissue in an in-vivo or in-vitro experimental condition for a biological tissue of a to-be-observed object, and can contribute to both diagnosis and photodynamic therapy.

Moreover, the present invention can provide a fluorescence detection and photodynamic therapy apparatus for animal experiment, which is simple in structure and inexpensive in manufacturing cost through supply of an image by a multispectral imaging unit having a one-chip multispectral sensor without a separate complex image processing work.

The invention has been described in detail with reference to preferred embodiments thereof. However, it will be appreciated by those skilled in the art that changes and modifications may be made in these embodiments without departing from the principles and spirit of the invention, the scope of which is defined in the appended claims and their equivalents. Therefore, what those skilled in the art to which the present invention pertains easily derive from the detailed description and the embodiment of the present invention should be construed as falling within the scope of the present invention.

What is claimed is:

1. A combined apparatus for detection of a multispectral optical image emitted from a living body and for light therapy, the apparatus comprising:
a combined light source unit including a plurality of coherent and non-coherent light sources configured to irradiate light onto a to-be-observed object while performing continuous illumination;
an optical imaging unit configured to form an image of the to-be-observed object and project the image to an image processing/controlling system;
a multispectral imaging unit including a one-chip multispectral sensor and the image processing/controlling system;
a blocking filter installed between the to-be-observed object and the one-chip multispectral sensor, the blocking filter being configured to block some light reflected off from the to-be-observed object while allowing some light and fluorescent light to pass therethrough;
a computer system configured to process, analyze, reproduce and store the image acquired from the multispectral imaging unit 30, and transfer the image to a display device and control the overall operation of all the related elements; and
the display device configured to display a processing result of the image by the computer system.

2. The apparatus according to claim 1, wherein the combined light source unit comprises a first light source, a second light source, and a third light source.

3. The apparatus according to claim 2, wherein the first light source 11 is a white-light source emitting light in a wavelength of 400 nm to 700 nm.

4. The apparatus according to claim 2, wherein the second light source is a monochrome light source consisting of two laser light sources.

5. The apparatus according to claim 2, wherein the third light source is a band-pass light source including a lamp emitting light in a short wavelength range.

6. The apparatus according to claim 3, wherein the white-light source is any one selected from the group consisting of a halogen lamp, a white lamp, an RGB LED, a xenon lamp, and a metal haloid lamp.

7. The apparatus according to claim 4, wherein the laser light source is any one selected from the group consisting of a single laser diode, a plurality of laser diode arrays, and a fiber-pigtailed laser diode, each of which emits monochrome light in a wavelength of from 400 nm to 900 nm.

8. The apparatus according to claim 5, wherein the band-pass light source is any one selected from the group consisting of a mercury lamp, an LED, a fiber-pigtailed LED, and a xenon lamp, each of which includes a band-pass filter having a half-intensity width of 60 nm or less in a wavelength range of from 320 nm to 600 nm

9. The apparatus according to claim 1, further comprising a light guide serving as a common irradiation path of light emitted from the first light source, the second light source, and the third light source.

10. The apparatus according to claim 9, wherein the second light source and the third light source irradiate light onto the to-be-observed object through the common light guide, and the first light source irradiates light onto the to-be-observed object directly, but not through the common light guide.

11. The apparatus according to claim 10, wherein the second light source and the third light source irradiate light onto the to-be-observed object through different light guides.

12. The apparatus according to claim 9, wherein the common light guide is a liquid light guide.

13. The apparatus according to claim 1, wherein a first mirror is disposed in front of the first light source of the combined light source unit to allow light emitted from the first light source to be reflected therefrom toward the liquid light guide.

14. The apparatus according to claim 13, wherein the first mirror is a dichroic mirror and is arranged so as be moved toward the first light source or the second light source by a certain driving means to allow light from the first light source and light from the second light source to be alternately irradiated onto the light guide.

15. The apparatus according to claim 1, wherein a second mirror is disposed in front of the second light source of the combined light source unit to allow lights emitted from two lasers to be simultaneously irradiated onto the common liquid light guide.

16. The apparatus according to claim 15, wherein a focal lens is further disposed in front of the second mirror to allow light emitted from the second light source to be irradiated onto the common light guide.

17. The apparatus according to claim 15, wherein the second mirror is a dichroic mirror.

18. The apparatus according to claim 5, wherein the band-pass filter of the band-pass light source as the third light source is arranged in plural numbers along a circumferential direction within a filter wheel rotatably driven by a given driving source for the rapid exchange of a filter.

19. The apparatus according to claim 5, wherein the band-pass filter of the band-pass light source as the third light source is either a single band-pass filter or a multi-band-pass filter.

20. The apparatus according to claim 1, wherein a projective lens is installed between the liquid light guide allowing light from the light sources of the combined light source unit to entering therethrough and the to-be-observed object to allow light irradiation to be performed on the to-be-observed object by uniformly magnifying light.

21. The apparatus according to claim 1, wherein a movable polarizer for operation under a crossed polarized light condition is installed between the liquid light guide allowing light from the light sources of the combined light source unit to entering therethrough and the to-be-observed object.

22. The apparatus according to claim 11, wherein the light guide for light irradiation of different paths is a laser light guide using a monofiber.

23. The apparatus according to claim 22, wherein a collimating lens is additionally installed behind the monofiber light guide to allow light to be irradiated onto a narrower site of the to-be-observed object side.

24. The apparatus according to claim 1, wherein the optical imaging unit is any one selected from the group consisting of an objective lens, an endoscope and a stereo microscope.

25. The apparatus according to claim 24, wherein the objective lens has a fixed focal point.

26. The apparatus according to claim 24, wherein the objective lens a zoom function.

27. The apparatus according to claim 24, wherein the objective lens has an automatic focusing function performed by a motor.

28. The apparatus according to claim 24, wherein the objective lens has an aperture stop for controlling the quantity of light and the depth of field.

29. The apparatus according to claim 1, wherein the one-chip multispectral sensor is a one-chip image sensor, which has light sensitivity in visible light and near-infrared wavelength ranges and has a mosaic-like arrangement formed by an R-canal filter, a G-canal filter, and a B-canal filter.

30. The apparatus according to claim 1, wherein the one-chip multispectral sensor is a one-chip image sensor, in which since each of the red, green and blue spectral filters has an additional pass band in a visible light (VIS) wavelength range as well as a near-infrared (NIR) wavelength range, all the pixels have a light sensitivity in the visible light wavelength range as well as in the near-infrared wavelength range.

31. The apparatus according to claim 29, wherein the one-chip image sensor is a CCD image sensor.

32. The apparatus according to claim 29, wherein the one-chip image sensor is a CMOS image sensor.

33. The apparatus according to claim 29, wherein the one-chip image sensor is an EMCCD.

34. The apparatus according to claim 1, wherein the blocking filter is any one selected from the group consisting of a single-band-pass filter, a multi-band-pass filter, a notch filter, and an edge long pass filter.

35. The apparatus according to claim 34, wherein the blocking filter is arranged in plural numbers along a circumferential direction within a filter wheel rotatably driven by a given driving source for the rapid exchange of a filter.

36. The apparatus according to claim 1, wherein the multispectral imaging unit 30 comprises an image processing/controlling system for controlling the one-chip multispectral sensor, and is provided to simultaneously acquire an image of a biological tissue as the to-be-observed object by formation of a multispectral image under the condition of fluorescence and reflected light or two fluorescences in which excitation lights are different in wavelength.

37. The apparatus according to claim 1, wherein the display device is an RGB monitor.

38. The apparatus according to claim 5, wherein the band-pass light source as the third light source emits light with a wavelength range of from 370 nm to 410 nm, and are used to simultaneously excite several fluorophores (NADH, Flavin and Porphyrin) along with the laser as the second light source.

39. The apparatus according to claim 4, wherein the laser as the second light source emits light with a wavelength range of 635 nm, and are used to simultaneously excite several fluorophores (NADH, Flavin and Porphyrin) along with the band-pass light source as the third light source.

40. The apparatus according to claim 3, wherein the laser (805 nm) as the second light source is used to excite indocyanine green while the white light source as the first light source emits polarized light.

41. The apparatus according to claim 1, wherein the optical imaging unit, the blocking filter, and the multispectral imaging unit including the one-chip multispectral sensor and the image processing/controlling system are integrally assembled in a single imaging head, and the imaging head is ascendably and descendably installed at a certain support.

42. The apparatus according to claim 40, wherein the support 82 comprises a vertical support assembled allow the imaging head 80 to ascend and descend, and a horizontal support integrally joined at a side thereof to a lower end of the vertical support to allow the to-be-observed object to be placed on the horizontal support, so that the imaging head can be moved in a horizontal direction relative to an optical axis of the imaging head so as to be focused on the to-be-observed object placed on the horizontal support.

43. The apparatus according to claim 8, wherein the band-pass filter of the band-pass light source as the third light source is arranged in plural numbers along a circumferential direction within a filter wheel rotatably driven by a given driving source for the rapid exchange of a filter.

44. The apparatus according to claim 8, wherein the band-pass filter of the band-pass light source as the third light source is either a single band-pass filter or a multi-band-pass filter.

45. The apparatus according to claim 9, wherein a projective lens is installed between the liquid light guide allowing light from the light sources of the combined light source unit to entering therethrough and the to-be-observed object to allow light irradiation to be performed on the to-be-observed object by uniformly magnifying light.

46. The apparatus according to claim 9, wherein a movable polarizer for operation under a crossed polarized light condition is installed between the liquid light guide allowing light from the light sources of the combined light source unit to entering therethrough and the to-be-observed object.

47. The apparatus according to claim 29, wherein the one-chip multispectral sensor is a one-chip image sensor, in which since each of the red, green and blue spectral filters has an additional pass band in a visible light (VIS) wavelength range as well as a near-infrared (NIR) wavelength range, all the pixels have a light sensitivity in the visible light wavelength range as well as in the near-infrared wavelength range.

48. The apparatus according to claim 8, wherein the band-pass light source as the third light source emits light with a wavelength range of from 370 nm to 410 nm, and are used to simultaneously excite several fluorophores (NADH, Flavin and Porphyrin) along with the laser as the second light source.

49. The apparatus according to claim 7, wherein the laser as the second light source emits light with a wavelength range of 635 nm, and are used to simultaneously excite several fluorophores (NADH, Flavin and Porphyrin) along with the band-pass light source as the third light source.

50. The apparatus according to claim 6, wherein the laser (805 nm) as the second light source is used to excite indocyanine green while the white light source as the first light source emits polarized light.
